(19) United States
(12) Patent Application Publication
(10) Pub. No.: US 2026/0215977 A1
(43) Pub. Date: Jul. 30, 2026

(54) ABSORBENT MEDICAL SHEET ASSEMBLIES

(71) Applicant: Sage Products, LLC, Cary, IL (US)

(72) Inventors: Emma KHAN, Chicago, IL (US); Brian ECKLUND, McHenry, IL (US); Abhishek PATEL, North Barrington, IL (US); Michael HIMES, Arlington Heights, IL (US); David BECK, Crystal Lake, IL (US)

(21) Appl. No.: 19/451,104

(22) Filed: Jan. 16, 2026

Related U.S. Application Data
(60) Provisional application No. 63/749,236, filed on Jan. 24, 2025.

(51) Int. Cl. A61F 13/84; A61F 13/15; A61F 13/511; A61F 13/514

(52) U.S. Cl. CPC A61F 13/8405; A61F 13/511; A61F 13/514; A61F 2013/15056; A61F 2013/15146; A61F 2013/15154; A61F 2013/8411

(57) ABSTRACT

An absorbent medical sheet assembly includes an impermeable layer and a permeable layer coupled to the impermeable layer. The permeable layer is in confronting relation to the impermeable layer and cooperating with the impermeable layer to define a cavity therebetween. The absorbent medical sheet includes an absorbent dam positioned between the impermeable layer and the permeable layer such that the cavity extends around an entirety of the absorbent dam.

### CROSS-REFERENCE TO RELATED APPLICATION

This Patent Application claims the benefit of and priority to U.S. Provisional Patent Application No. 63/749,236, filed Jan. 24, 2025, the entire disclosure of which is hereby incorporated by reference herein.

### TECHNICAL FIELD

The present disclosure relates generally to absorbent medical sheet assemblies. In particular, this application relates to systems and methods for absorbing medical waste.

### BACKGROUND

Patients in medical facilities often suffer from various ailments or physical limitations. Such ailments or limitations commonly restrict patients from using a traditional toilet system when expelling biological waste. Some patients are not sufficiently mobile to self-transfer to a traditional toilet system. Other patients may suffer from incontinence and therefore are unable to control expulsion of their bodily waste.

### SUMMARY

One embodiment of the present disclosure relates to an absorbent medical sheet assembly including an impermeable layer, and a permeable layer coupled to the impermeable layer. The permeable layer is in confronting relation with the impermeable layer and cooperates with the impermeable layer to define a cavity therebetween. The absorbent medial sheet assembly includes an absorbent dam positioned between the impermeable layer and the permeable layer such that the cavity extends around an entirety of the absorbent dam.

In some embodiments, the absorbent dam has a first volume in an unsaturated state and a second volume in a saturated state, the second volume being larger than the first volume.

In some embodiments, the absorbent dam includes a first segment extending along a first axis and a second segment extending at least in part along a second axis. The second axis is angularly separated from the first axis by an angle between 45-135°.

In some embodiments, the permeable layer is defined by a permeable layer width extending from a permeable layer first edge to a permeable layer second edge, and a permeable layer length extending from a permeable layer third edge to a permeable layer fourth edge. The first segment of the absorbent dam has a dam width that is 0.2-0.8× the permeable layer width

In some embodiments, the second segment of the absorbent dam has a dam length that is 0.2-0.8× the permeable layer length.

In some embodiments, the second segment extends from the first segment at an angle of 85-95°.

In some embodiments, the first segment of the absorbent dam is contiguous with the second segment of the absorbent dam to form a curved connection between the first segment and the second segment.

In some embodiments, the first segment of the absorbent dam is noncontiguous with the second segment of the absorbent dam, the second segment of the absorbent dam separated from the first segment of the absorbent dam by 0.1-0.4× the permeable layer width.

Another embodiment of the present disclosure relates to a hand covering. The hand covering includes an impermeable liner including a liner interior surface defining a liner cavity within the impermeable liner, a liner exterior surface, a first liner edge defining a first opening into the liner cavity, and a second liner edge opposite the first liner edge. The second liner edge is sealed. The hand covering includes an absorbent sheet including a sheet interior surface defining a sheet cavity within the absorbent sheet, a sheet exterior surface, and a sheet edge defining a second opening into the sheet cavity. The impermeable liner is positioned within the sheet cavity, the sheet interior surface being coupled to at least a portion of the liner exterior surface. The hand covering includes a gusset extending into the liner cavity.

In some embodiments, the hand covering further includes a rigid scraper coupled to the sheet exterior surface. The rigid scraper extends into the gusset.

In some embodiments, the hand covering includes a sealed, frangible enclosure positioned between the impermeable liner and the absorbent sheet. The sealed, frangible enclosure includes a fluid solution therein and adapted to break in response to an applied force exceeding a threshold force of 10-15 pounds.

In some embodiments, the absorbent sheet is impregnated with a pH-neutralizing agent.

In some embodiments, the absorbent sheet has a sheet length that is 0.25-0.75× a liner length.

Another embodiment of the present disclosure relates to an absorbent medical sheet assembly including an impermeable layer, an absorbent layer coupled to the impermeable layer, and an absorbent dam positioned on the absorbent layer. The absorbent dam includes a first segment extending along a first axis, and a second segment extending at least in part along a second axis, The second axis angularly separated from the first axis. The first segment and the second segment define a region therebetween configured to retain biological waste.

In some embodiments, the first segment is noncontiguous with the second segment and defines a first breach therebetween.

In some embodiments, the absorbent dam further includes a third segment extending at least in part along a third axis, the third axis angularly separated from the first axis, the first segment being noncontiguous with the third segment and defining a breach therebetween.

In some embodiments, the absorbent dam is a first absorbent dam, and the absorbent medical sheet assembly further includes a second absorbent dam distal to the first absorbent dam and configured to receive biological waste through the first breach from the region.

In some embodiments, the absorbent medical sheet assembly further includes a first angled absorbent dam positioned on the absorbent layer, and a second angled absorbent dam positioned on the absorbent layer a distance away from the first angled absorbent dam. The first angled absorbent dam and the second angled absorbent dam configured to direct the biological waste therebetween and into the region between the first segment and the second segment.

In some embodiments, the second angled absorbent dam extends from the first angled absorbent dam at an angle between 45-135°.

In some embodiments, the first segment is contiguous with the second segment to form a curved connection between the first segment and the second segment.

### BRIEF DESCRIPTION OF THE DRAWINGS

The details of one or more implementations are set forth in the accompanying drawings and the description below. Other features, aspects, and advantages of the disclosure will become apparent from the description, the drawings, and the claims, in which:

FIG. 1 is a top view of an example absorbent medical sheet assembly;

FIG. 2 is a top view of an impermeable layer of the absorbent medical sheet assembly of FIG. 1;

FIG. 3 is a top view of another example absorbent medical sheet assembly;

FIG. 4 is a side cross-sectional view of the absorbent medical sheet assembly of FIG. 1 in an unsaturated state;

FIG. 5 is a side cross-sectional view of the absorbent medical sheet assembly of FIG. 1 in a saturated state;

FIG. 6 is a top cross-sectional view of the absorbent medical sheet assembly of FIG. 4;

FIG. 7 is a top cross-sectional view of the absorbent medical sheet assembly of FIG. 5;

FIG. 8 is a top view of another example absorbent medical sheet assembly;

FIG. 9 is a top view of another example absorbent medical sheet assembly;

FIG. 10 is a top view of another example absorbent medical sheet assembly;

FIG. 11 is a top view of an example hand covering;

FIG. 12 is a top view of another example hand covering;

FIG. 13 is a side cross-sectional view of the hand covering of FIG. 11;

FIG. 14 is a front view of an absorbent sheet of the hand covering of FIG. 11;

FIG. 15 is a front view of a liner of the hand covering of FIG. 11;

FIG. 16 is a side cross-sectional view of the liner of FIG. 15;

FIG. 17 is a side cross-sectional view of the absorbent sheet of FIG. 14;

FIG. 18 is a front view of another example hand covering;

FIG. 19 is a side cross-sectional view of the hand covering of FIG. 18; and

FIG. 20 is another side cross-sectional view of the hand covering of FIG. 18.

### DETAILED DESCRIPTION

Following below are more detailed descriptions of various concepts related to, and implementations of, methods, apparatuses, and systems for a formulation application system. The various concepts introduced above and discussed in greater detail below may be implemented in any of a number of ways, as the described concepts are not limited to any particular manner of implementation. Examples of specific implementations and applications are provided primarily for illustrative purposes.

### I. Overview

In various situations, a patient may involuntarily expel bodily waste onto a support surface, such as a bed or seat, upon which the patient is positioned. This bodily waste may present a hazard to the patient, medical staff, and other patients. It is difficult to expeditiously and repeatably clean the bodily waste.

Traditional support surfaces, such as mattresses and seats, are not adapted to contain expelled bodily waste such as excrement (particularly liquid stool) and urine. The bodily waste is absorbed into the support surface (e.g., mattress or seat), pools around the patient (e.g., around the back, gluteal region, and legs), and/or disperses in an uncontrolled manner (e.g., toward the legs, onto an unprotected sheet, onto the floor) causing injuries, waste, and unsafe conditions. To provide protection for the support surface, non-absorbent, non-permeable pads may be placed under the patient. However, these traditional non-absorbent pads do not sufficiently contain the bodily waste to the excretion area (e.g., the gluteal area), thus allowing the bodily waste to spread down the legs of the patient and onto the mattress or floor.

This spread of bodily waste results in soiled bedding, increased spread of contamination (e.g., bacteria, viruses, and parasites), unsafe working conditions (e.g., slip hazards on the floor near the bed), and increased potential of incontinent-associated dermatitis (“IAD”) and pressure injury. After an incontinence episode, as described above, it may take anywhere from 20-30 minutes to fully change the linen of the bedding, increasing medical costs, putting a strain on nursing staff, and posing risks to patient and nurse safety due to moving the patient during the change. Some impermeable pads include an absorbent layer, allowing for liquefied stool and urine to soak into the pad to protect the mattress or seat. However, traditional absorbent pads do not absorb the liquid waste at a fast enough rate during large expelling events, leading to the same challenges described above.

In addition to containing bodily waste during an incontinence episode, patients in medical facilities may additionally or alternatively require assistance in cleaning the expelled bodily waste after an incontinence episode, particularly in the patient's skin folds and/or crevices (e.g., the gluteal and/or the groin area). Traditionally, this may involve a nurse aiding the patient by cleaning the patient and surrounding areas with one-time-use towels. These one-time-use towels have several deficiencies, such as only being able to use one side of the towel during cleaning, exposing medical personnel to bodily waste and potential biohazards, and not fully containing the collected/cleaned bodily waste after clean up.

Systems and methods described herein may be used to prevent and/or control the spread of biological waste, such as excrement, urine, sweat, exudate, and/or blood during or after an incontinent episode (e.g., an expelling event, waste elimination, expelling). As described in greater detail herein, a sheet assembly containing one or more absorbent dams may be positioned under a patient on a support surface (e.g., a bed, an examination table, a seat, a wheelchair, a surgical table) to prevent and/or control the spread of the expelled biological waste during absorption of the expelled biological waste into the pad.

Additionally or alternatively, an absorbent hand covering (e.g., a mitten, sleeve, and/or glove) having a scraper coupled thereto may be used during the cleaning of the expelled biological waste to further prevent and/or contain the spread of the expelled biological waste. In at least one implementation, the systems and methods described herein may be used in a medical/care facility. As used herein, biological waste (e.g., biological fluids, expelled waste, eliminated waste, expelled biological wastes) may include, but is not limited to, fecal matter (solid or liquid), urine, blood, sweat, exudate, and/or saliva.

### II. Overview of Absorbent Medical Sheet Assembly

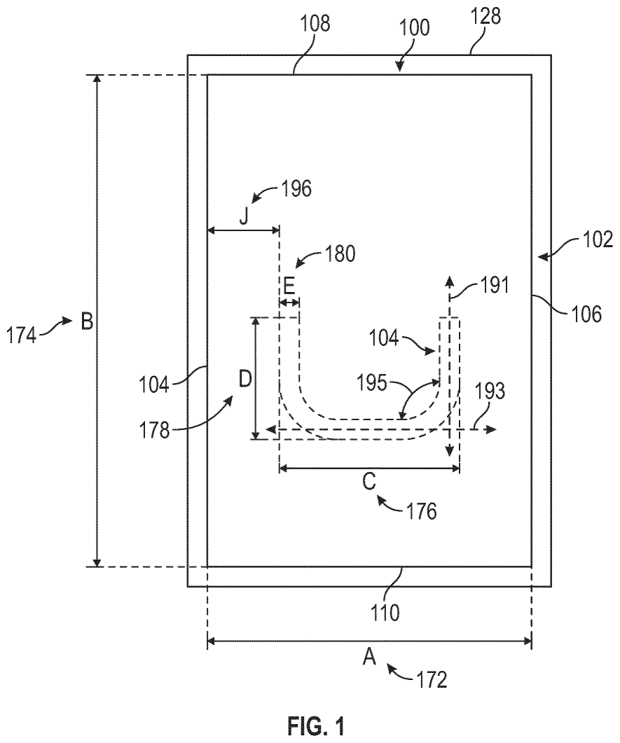

In a first implementation of the systems and methods described herein, as shown in FIGS. 1-10, a medical sheet assembly may be positioned under a patient on a support surface. The medical sheet assembly is utilized to mitigate and/or control spread of biological waste expelled by the patient through use of one or more absorbent dams selectively positioned within the medical sheet assembly. The selectively positioned absorbent dams expand during absorption of fluid. Thus, during an event where biological waste is expelled, the selectively positioned absorbent dams may absorb the expelled biological waste, expand in volume, and provide a raised structure that causes the biological waste to pool (pooled biological waste) and thereby mitigate or prevent the spread of the pooled biological waste to other parts of the medical sheet assembly, support surface, floor, or parts of the patient. This absorbent dam maintains the pooled biological waste in a predefined area with additional absorbent material underneath—or adjacent—to absorb the pooled biological waste. The damming action provides the additional absorbent sufficient material time to completely absorb the pooled biological waste until, for example, full saturation.

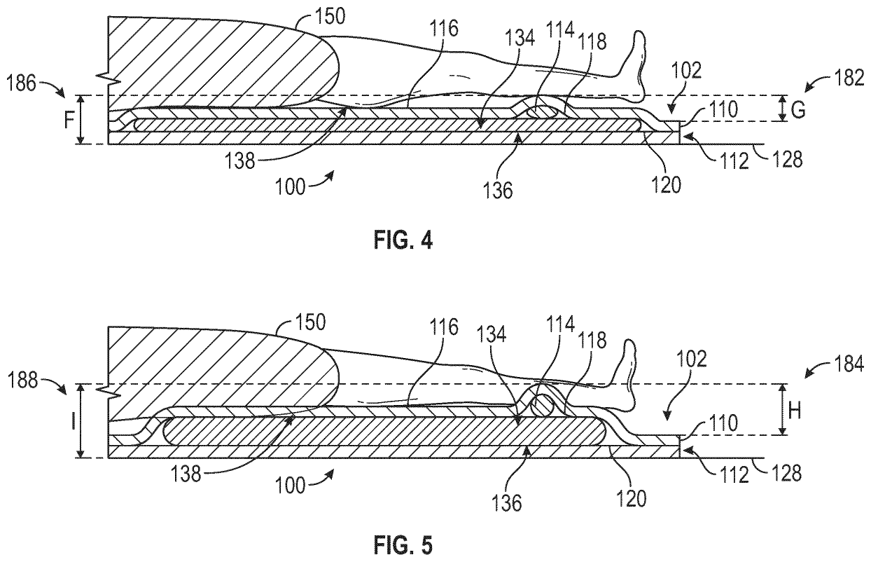

FIG. 1 depicts an absorbent medical sheet assembly 100 (e.g., an absorbent pad, non-woven fabric sheet, sheet assembly, disposable pad), according to various embodiments. The absorbent medical sheet assembly 100 is a multi-layered assembly of various components and sheets configured to provide one or more selectively deployed absorbent dams to mitigate the spread of expelled biological waste from a patient. As is described in more detail herein, the absorbent medical sheet assembly 100 provides a protective barrier between a patient 150 (as shown in FIG. 4) and a support surface 128 (e.g., a bed, an examination table, a seat, a wheelchair, a surgical table). The support surface 128 may be any surface upon which the patient 150 is positioned and supported, either in a seated or reclined position (e.g., prone, supine). The absorbent medical sheet assembly 100 may act as a barrier to protect the support surface 128 from expelled biological waste of the patient 150. The absorbent medical sheet assembly 100 may also act as a barrier to protect the patient 150 from contaminants on the support surface 128, providing a sterile environment for the patient 150.

The absorbent medical sheet assembly 100 includes a permeable layer 102 (e.g., scrim, top sheet, first sheet, non-woven sheet, wicking sheet) that contacts the patient 150. For example, a back of the patient 150 contacts the permeable layer 102 when the patient 150 is lying (e.g., in a prone position) on the absorbent medical sheet assembly 100. In some applications, the patient is placed on the permeable layer 102 by a caretaker (e.g., nurse, doctor, technician).

The permeable layer 102 facilitates passage of liquids (e.g., liquid stool, urine) through the permeable layer 102 to lower components and/or layers of the absorbent medical sheet assembly 100. The permeable layer 102 may also be configured to provide additional benefits, including absorbency, liquid repellence, resilience, stretching capability, softness, enhanced strength, flame retardancy, washability, cushioning, thermal insulation, filtration, and anti-bacterial capability. The permeable layer 102 may have a hydrophilic treatment (e.g., the permeable layer 102 may be coated with a hydrophilic coating) that allows for the permeable layer 102 to remain dry to the touch after being exposed to biological waste, while the structure (e.g., non-woven) of the permeable layer 102 facilitates passage of biological waste to pass through to additional layers of the absorbent medical sheet assembly 100 underneath the permeable layer 102.

The permeable layer 102 is made of a non-woven, fabric-like material of fibers bonded through at least one chemical process, mechanical process, thermal process, or solvent process. In various embodiments, the permeable layer 102 is made of non-woven polyester. However, the permeable layer 102 may be made from various other non-woven materials such as spunbond non-woven materials (e.g., polyethylene fiber materials, polypropylene fiber materials), meltblown non-woven materials, spunbond meltblown spunbond (SMS) non-woven materials, needle punched non-woven materials (e.g., wool, cotton, synthetic fibers), bonded non-woven materials, coated non-woven materials (e.g., a layer of spunbond non-woven fibers coated with synthetic fibers or plastic), laminated non-woven materials, thermal bonded non-woven materials, drylaid non-woven materials, spunlaid non-woven materials, or wetlaid non-woven materials. The permeable layer 102 may be made from, for example, polymers, polyester super absorbing powder (SAP), water-soluble binders, ceramic-impregnated materials, geotextiles, or other similar materials. The permeable layer 102, in some embodiments, is made from, polyurethane foam, knitted fabrics, woven fabrics, natural fibers, polyester blends, cotton, silk, perforated films, and/or mesh fibers.

The permeable layer 102 may, in some embodiments, act as an acquisition layer, which acts to distribute and channel expelled biological waste away from an exterior surface of the permeable layer 102 for even absorption into one or more underlying absorbent components (e.g., an absorbent layer, an absorbent dam) of the absorbent medical sheet assembly 100, as described in more detail below. In some embodiments, the absorbent medical sheet assembly 100 includes a separate acquisition layer between the permeable layer 102 and the one or more underlying absorbent components.

The permeable layer 102 reduces strike-back of biological waste once the biological waste is absorbed into the absorbent medical sheet assembly 100 (e.g., absorbed into the absorbent layer and/or the absorbent dam). As used herein, strike-back of biological waste refers to the passage of fluid from an interior of the absorbent medical sheet assembly 100 back through the permeable layer 102 to an exterior surface of the permeable layer 102. This strike-back resistance mitigates the release of absorbed biological waste from the one or more underlying absorbent components (e.g., the absorbent layer and/or the absorbent dam) back out to the exterior surface of the permeable layer 102. Rather, the biological waste is retained by the one or more underlying absorbent components of the absorbent medical sheet assembly 100 once absorbed by the one or more underlying absorbent components of the absorbent medical sheet assembly 100.

The permeable layer 102 includes a permeable layer first edge 104 and a permeable layer second edge 106 opposite the permeable layer first edge 104. The permeable layer 102 also includes a permeable layer third edge 108 that extends between the permeable layer first edge 104 and the permeable layer second edge 106. The permeable layer 102 also includes a permeable layer fourth edge 110 that is opposite the permeable layer third edge 108. The permeable layer fourth edge 110 extends between the permeable layer first edge 104 and the permeable layer second edge 106. The permeable layer third edge 108 and/or the permeable layer fourth edge 110 defines a permeable layer width A 172. The permeable layer first edge 104 and/or the permeable layer second edge 106 defines a permeable layer length B 174. The permeable layer 102 has a peripheral edge including the permeable layer first edge 104, the permeable layer second edge 106, the permeable layer third edge 108, and the permeable layer fourth edge 110. The permeable layer 102 may be formed in other shapes and include different configurations of its peripheral edge.

The permeable layer 102 includes a permeable exterior surface 116 that contacts the patient. The permeable layer 102 also includes a permeable interior surface 118 opposite the permeable exterior surface 116.

In some embodiments (as shown in FIG. 1), the absorbent medical sheet assembly 100 is defined by the permeable layer width A 172 of 2-8 feet. The absorbent medical sheet assembly 100 may also be defined by the permeable layer length B 174 of 2-8 feet. Relative to the permeable layer width A 172 and/or the permeable layer length B 174, the absorbent dam 114 has a dam width C 176 that is 0.2-0.8× the permeable layer width A 172. The absorbent dam 114 has a dam length D 178 that is 0.2-0.8× the permeable layer length B 174. The absorbent dam 114 has a dam thickness E 180 that is 0.1-0.25× the permeable layer width A 172. In some embodiments, instead of being defined by dimensions of the permeable layer, the absorbent medical sheet assembly 100 is defined by an impermeable layer width and an impermeable layer length.

In some embodiments, the absorbent medical sheet assembly 100 includes handles along at least one of the permeable layer first edge 104, the permeable layer second edge 106, the permeable layer third edge 108, or the permeable layer fourth edge 110. The handles may be die-cut out of the permeable layer 102 and/or an impermeable layer of the absorbent medical sheet assembly 100 (as described herein). The handles may be reinforced using welding, stitching, adhesive, or other joining methods. Likewise, the handles may be reinforced using injection/compression molded handles welded between the permeable layer 102 and the impermeable layer, or otherwise coupled to the permeable layer 102 and/or the impermeable layer (e.g., attached above or below). The handles may be reinforced by adding additional or alternative layers of woven material, non-woven material, or film. The reinforcement layers may be sewn, welded, adhered, or joined using other various methods. Alternatively, the material of the permeable layer 102 and/or the impermeable layer may be folded to increase strength or create handles. In some embodiments, webbing is attached to one or more of the permeable layer first edge 104, the permeable layer second edge 106, the permeable layer third edge 108, and/or the permeable layer fourth edge 110 to create handles.

The permeable layer 102 may have moisture-wicking properties such that the permeable layer 102 wicks moisture from the patient and transports the wicked moisture into the absorbent medical sheet assembly 100 to lower layers (e.g., an impermeable layer of the absorbent medical sheet assembly 100 and/or an absorbent dam of the absorbent medical sheet assembly 100). The permeable layer 102 may not only wick moisture into the interior of the absorbent medical sheet assembly 100, it may also wick moisture laterally along the permeable exterior surface 116 such that moisture (e.g., expelled biological waste) is disbursed throughout the permeable layer 102 to the components of the absorbent medical sheet assembly 100 beneath the permeable layer 102.

The non-woven material of the permeable layer 102 may include one or more channels through which the wicked moisture may travel (e.g., through capillary action). The channels may be formed by interstitial spacing between non-woven fibers of the permeable layer 102.

The absorbent medical sheet assembly 100 includes an impermeable layer 112 (e.g., bottom layer, second layer, outer layer, back sheet). The impermeable layer 112 may be adapted such that biological waste does not pass through the impermeable layer 112, thereby providing a barrier between the expelled biological waste and the support surface 128.

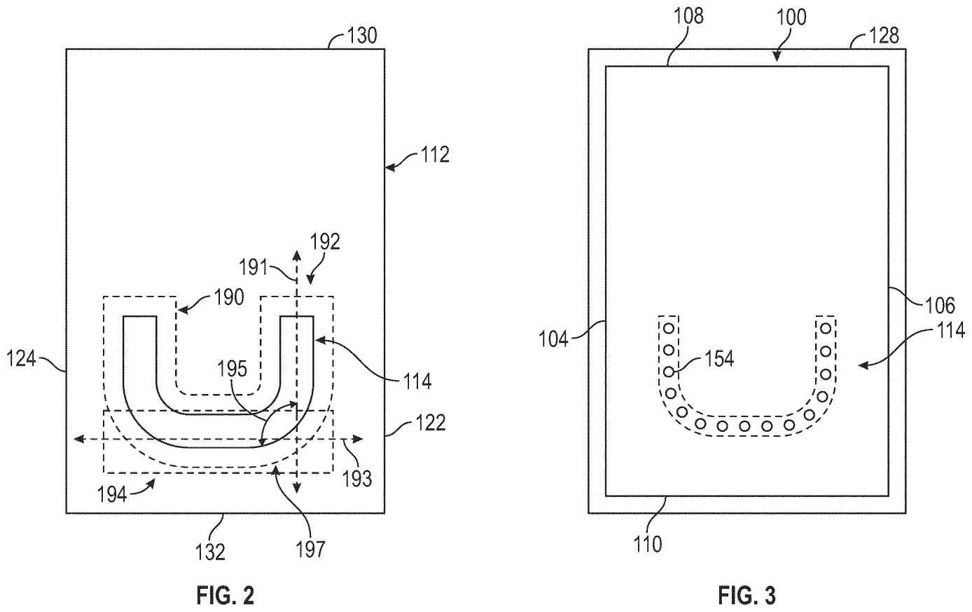

As shown in FIG. 2, the impermeable layer 112 includes an impermeable layer first edge 122 and an impermeable layer second edge 124 opposite the impermeable layer first edge 122. The impermeable layer 112 also includes an impermeable layer third edge 130 that extends between the impermeable layer first edge 122 and the impermeable layer second edge 124. The impermeable layer 112 also includes an impermeable layer fourth edge 132 that is opposite the impermeable layer third edge 130. The impermeable layer fourth edge 132 extends between the impermeable layer first edge 122 and the impermeable layer second edge 124. The impermeable layer third edge 130 and/or the impermeable layer fourth edge 132 defines an impermeable layer width. The impermeable layer width may be 95-105% the permeable layer width. The impermeable layer first edge 122 and/or the impermeable layer second edge 124 define an impermeable layer 112 length. The impermeable layer length may be 95-105% a permeable layer length B 174. The impermeable layer 112 has a peripheral edge including the impermeable layer first edge 122, the impermeable layer second edge 124, the impermeable layer third edge 130, and the impermeable layer fourth edge 132. The impermeable layer 112 may be formed in other shapes and include different configurations of its peripheral edge. The impermeable layer 112 may be geometrically similar to the permeable layer 102 regardless of the shape of the permeable layer, such that the impermeable layer has the same shape of the of the permeable layer 102 with substantially the same dimensions (e.g., 95-105% the dimensions of the permeable layer).

The impermeable layer 112 may have an impermeable layer interior surface 120 (as shown in FIGS. 4 and 5) in confronting relation with the permeable interior surface 118 at one or more locations. In an embodiment, the permeable layer 102 is coupled to the impermeable layer 112 along a peripheral edge of the permeable layer 102. For example, the permeable layer first edge 104 may be in confronting relation with impermeable layer first edge 122, the permeable layer second edge 106 may be in confronting relation with the impermeable layer second edge 124, the permeable layer third edge 108 may be in confronting relation with the impermeable layer third edge 130, and/or the permeable layer fourth edge 110 may be in confronting relation with the impermeable layer fourth edge 132. The impermeable layer interior surface 120 may be coupled to the permeable interior surface 118 through one or more joining methods, such as sewing, welding, hot melt adhesive, heat sealing, and/or ultrasonic sealing.

The impermeable layer 112 and the permeable layer 102 cooperate together to define a liner cavity 134 therebetween. As is explained in more detail herein, the liner cavity 134 is utilized for housing one or more absorbent components of the absorbent medical sheet assembly 100 such that the biological waste absorbed by these absorbent components is contained between the permeable layer 102 and the impermeable layer 112.

The liner cavity 134 may have a single compartment or multiple compartments. For example, the permeable layer 102 may only be coupled to the impermeable layer 112 along a peripheral edge of the permeable layer 102 and a peripheral edge of the impermeable layer 112 (or along the entirety of the absorbent dam, as described herein), thus cooperating together to define the liner cavity 134 therebetween as a single compartment. However, the permeable layer 102 and the impermeable layer 112 may be in confronting relation with each and coupled together at locations in addition to—or alternative to—the peripheral edge (e.g., along lines parallel to the permeable layer first edge 104 and/or permeable layer third edge 108 or at points or locations within the peripheral edge). Such embodiments may reduce separation between the permeable layer 102 and the impermeable layer 112. In such embodiments, the permeable layer 102 and the impermeable layer 112 cooperate together to define the liner cavity 134 therebetween with multiple partitioned or segmented compartments.

The absorbent medical sheet assembly 100 also includes an absorbent layer 136 (e.g., an absorbent core, a super absorbent core, super absorbent layer, absorbent interior), as shown in FIGS. 4 and 5. The absorbent layer 136 absorbs and retains biological waste (e.g., expelled biological waste) in the liner cavity 134. In some embodiments, the absorbent layer 136 mitigates release of the absorbed biological waste from the absorbent layer 136 even under pressure. The absorbent layer 136 may be positioned in part or whole within the liner cavity 134 defined by the permeable interior surface 118 and the impermeable layer interior surface 120.

In some embodiments, the absorbent layer 136 is free to move within the liner cavity 134 in some embodiments. For example, the absorbent layer 136 may be contained between the permeable layer 102 and the impermeable layer 112 while not being coupled to the permeable layer 102 or the impermeable layer 112.

In some embodiments, the absorbent layer 136 is coupled to at least one of the permeable layer 102 (e.g., at the permeable interior surface 118) or impermeable layer 112 (e.g., the impermeable layer interior surface 120). This coupling may be by one or more bonding methods and/or techniques. For example, the absorbent layer 136 positioned in the liner cavity 134 may be coupled to the permeable layer 102 by adhesive bonding, thermal bonding, ultrasonic bonding, lamination, and/or mechanical bonding (e.g., stitching, crimping, embossing).

In some embodiments, the permeable layer 102 and/or the impermeable layer 112 cooperate with the absorbent layer 136 to maintain the absorbent layer 136 in position within the liner cavity 134 therebetween. For example, in bonding the absorbent layer 136 to the permeable layer 102 and/or the impermeable layer 112, the absorbent layer 136 is maintained flat in the liner cavity 134 defined by the layers without shifting within the liner cavity 134.

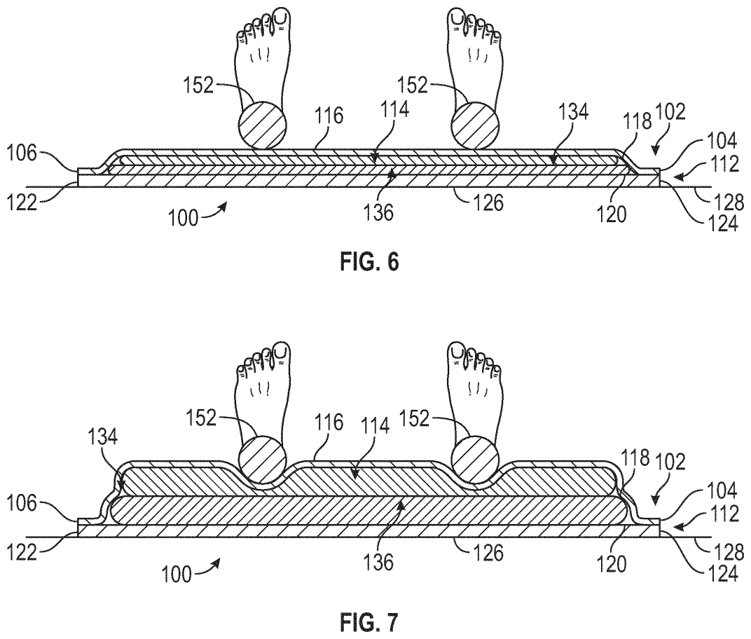

The absorbent layer 136 may have a first volume in an unsaturated state (shown in FIGS. 4 and 6) and a second, larger volume in the saturated state (shown in FIGS. 5 and 7). The unsaturated state may refer to a dry condition in which the absorbent layer 136 has a low to no moisture content (e.g., before absorbing expelled biological waste). The saturated state may refer to a wet condition in which the absorbent layer 136 has high moisture content (e.g., after absorbing expelled biological waste). The moisture content may refer to an absolute volume of fluid absorbed into the absorbent dam 114 (e.g., 100 ml, 500 ml, 1000 ml). The moisture content may refer to an amount of volume of absorbed fluid relative to a total amount of fluid capable of being absorbed by the absorbent dam 114 (e.g., 10%, 25%, 50%, 100%). The saturated state may occur after absorbing expelled biological waste, such as expelled fluid.

In some embodiments, the entirety of the liner cavity 134 within the coupled peripheral edges of the permeable layer 102 and the impermeable layer 112 is filled with at least a portion of the absorbent layer 136 to increase the absorption potential of the absorbent medical sheet assembly 100. In other embodiments, the absorbent layer 136 does not fill the entirety of the area between the coupled peripheral edges of the permeable layer 102 and the impermeable layer 112. In such embodiments, the absorbent layer 136 is located at specific locations of the absorbent medical sheet assembly 100, such as high-fluid-penetration areas, such as a region 138 of the absorbent medical sheet assembly 100 surrounding the gluteal region of the patient 150. Because a majority of expelled biological waste may emanate from the gluteal region of the patient 150, the absorbent layer 136 may be strategically positioned beneath this area for efficient absorption. In some embodiments, the absorbent layer 136 has additional absorbent material in the region 138 with lower amounts of absorbent material in surrounding areas.

The absorbent layer 136 may be comprised of a non-woven materials such as cellulose fibers, superabsorbent polymers (SAPs), or a combination of both, to provide high absorbency while ensuring biological waste retention even under pressure. The absorbent layer 136 may be coupled and/or bonded to the permeable layer 102 such that biological waste wicked into the absorbent medical sheet assembly 100 by the permeable layer 102 is quickly wicked away from the permeable exterior surface 116, keeping the skin of the patient 150 dry and reducing the risk of irritation or infection. The cellulose and/or SAP structure of the absorbent layer distributes the absorbed biological waste evenly through the absorbent layer 136, preventing over-saturation in specific areas.

In some embodiments, the absorbent layer 136 may include odor control to improve the comfort of the patient 150. For example, the absorbent layer 136 may be treated with a fluid solution with odor-neutralizing agents such as activated carbon, zeolites, baking soda, citric acid, and/or other derivatives therefrom. The treatments can include embedding the fluid solution with odor-neutralizing agents (e.g., activated carbon and/or zeolites) within the structure of the absorbent layer 136 and/or applying the odor-neutralizing agent to the absorbent layer 136 (e.g., baking soda and/or citric acid). The absorbent layer 136 may also be treated with fragrance or deodorants for reducing odor before, during, and/or after absorption. Some odor-neutralizing agents may be moisture-activated such that the odor-neutralizing properties are exhibited when the absorbent layer 136 is saturated.

Additionally or alternatively, the absorbent layer 136 may include antimicrobial properties to improve the hygiene of the patient 150. For example, the absorbent layer 136 may be treated with antimicrobial agents such as silver ions, zinc oxide, quaternary ammonium compounds, or a combination thereof. These compounds may inhibit bacterial or fungal growth. Other antimicrobial compounds that may be used to treat the absorbent layer 136 and reduce bacterial and/or fungal growth may include tea tree oil, aloe vera, chitosan, and/or a combination thereof.

The absorbent medical sheet assembly 100 may include an absorbent dam 114 (e.g., absorbent levy, absorbent barrier, deployable barrier, expandable barrier). The absorbent dam 114 provides a dynamically deployed dam/barrier to prevent the spread of expelled biological waste (e.g., solid stool, liquid stool, and/or urine). In some embodiments, the absorbent dam 114 is positioned such that it provides a dam around some or all of the region 138.

As described herein, the permeable layer 102 and the impermeable layer 112 may be in confronting relation and coupled about the peripheral edge, with the absorbent dam 114 being positioned therebetween within the liner cavity 134. The absorbent dam 114 may be placed within the liner cavity 134 such that the liner cavity 134 extends around an entirety of a periphery of the absorbent dam 114. The absorbent dam 114 may be positioned within the liner cavity 134 in addition or alternative to the absorbent layer 136.

The absorbent dam 114 may have a first volume in an unsaturated state (shown in FIGS. 4 and 6) and a second, larger volume in the saturated state (shown in FIGS. 5 and 7). The unsaturated state may refer to a dry condition in which the absorbent dam 114 has a low-to-no moisture content (e.g., before absorbing expelled biological waste). The saturated state having a larger volume may refer to a wet condition in which the absorbent dam 114 has a moisture content satisfying a moisture content threshold. The moisture content may refer to an absolute volume of fluid absorbed into the absorbent dam 114 (e.g., 100 ml, 500 ml, 1000 ml). The moisture content may refer to an amount of volume of absorbed fluid relative to a total amount of fluid capable of being absorbed by the absorbent dam 114 (e.g., 10%, 25%, 50%, 100%). The saturated state may occur after absorbing expelled biological waste, such as expelled fluid.

In the unsaturated state, the first volume of the absorbent dam 114 may lay substantially flat within the liner cavity 134 defined by the permeable layer 102 and the impermeable layer 112. By laying flat, the absorbent dam 114 does not increase pressure on body parts of the patient 150 (e.g., back, legs, gluteal region, arms, neck, feet, head). By laying flat and not increasing the pressure on the body parts of the patient 150, risk of pressure-related injuries (e.g., pressure ulcers, shear injuries, friction injuries, moisture-associated skin damage, deep tissue injury, ischemic injury, skin tears, maceration, nerve compression) is reduced.

In the saturated state, the absorbent dam 114 may be configured to dynamically expand (e.g., to the second volume) to provide a dam for the expelled biological waste. In the saturated state, the absorbent dam 114 no longer lays flat within the liner cavity 134. Rather, the absorbent dam 114 rises at least vertically, exerting a force on the permeable interior surface 118, causing the permeable layer 102 to rise away from the support surface 128. In some embodiments, this rising creates a dam for liquid, as shown in FIGS. 5 and 7. In some embodiments, the second volume is 1.5-10× the volume of the first volume. The absorbent dam 114 may expand not only in the vertical direction, but in all directions, as shown in FIGS. 5 and 7. The absorbent layer 136 may exhibit similar volume-changing characteristics as described with regard to the absorbent dam 114.

While the absorbent dam 114 is described as expanding when absorbing liquids, it should be understood that the systems and methods described herein extend to alternative methods of providing a dam around the region 138 of the patient 150 when upon the support surface 128. For example, alternative embodiments for the absorbent dam 114 may include an air cushion with an air bladder with an expandable cavity that, when filled with a gas, expands, thereby creating a dam. The air bladder is coupled to a valve (e.g., a one-way valve) that is fluidly coupled to an air pump for forcing a gas (e.g., air) into the air bladder to expand the air bladder. The pump is communicably coupled to a communication interface (e.g., a button, touch screen, switch, knob, sensor) that may be used to engage the air pump and initiate expansion of the air bladder. The communication interface may be included in a control panel or remote. In some embodiments, the communication interface is a sensor (e.g., a humidity/fluid/moisture sensor) that may send a signal to the air pump to initiate operation when the sensor senses moisture. For example, the sensor may be integrated into the region 138 and configured to sense moisture in the region 138. In some embodiments, the sensor is not integrated into the permeable layer 102, but rather placed positionally proximate (e.g., on top of) the region 138. While the sensor is described as being located at or near the region 138, it is understood that the sensor may be positioned anywhere along the permeable layer 102 or within the absorbent medical sheet assembly 100.

FIGS. 6 and 7 illustrate a cross-sectional front view of the absorbent medical sheet assembly 100. As shown in FIG. 6, in the unsaturated state, the absorbent dam 114 lays substantially flat within the liner cavity 134. In the saturated state, as shown in FIG. 7, the absorbent dam 114 expands to form a dam with one or more body parts 152 of the patient 150. The body parts 152 may include any body part, such as a leg (as shown in FIGS. 6 and 7), a back, a knee, an ankle, a foot, an arm, a neck, a posterior pelvic region, a buttocks, or a combination thereof. The absorbent dam 114 expands around the one or more body parts 152 to prevent the expelled biological waste from migrating down or up the absorbent medical sheet assembly 100 away from the region 138. This provides additional time for the absorbent layer 136 to absorb the expelled biological waste before migration of the expelled biological waste to other parts of the absorbent medical sheet assembly 100 or the support surface 128.

In some embodiments, the absorbent dam 114 and/or the permeable layer 102 around the absorbent dam 114 may be configured to expedite the absorption of the expelled biological waste into the absorbent dam 114, such that it absorbs and/or expands at a rate faster than the absorbent layer 136. For example, the absorbent dam 114 may be made of an absorbent material that absorbs biological waste at a faster rate than the absorbent layer 136. In some embodiments (as shown in FIG. 3), the permeable layer 102 includes perforations (e.g., aperture) within the permeable layer 102 to allow for faster ingress into the absorbent dam 114 from the exterior of the permeable layer 102. The perforations may include holes (as shown as hole 154 in FIG. 3) and/or slits. Alternatively or additionally, the permeable layer 102 around the absorbent dam 114 may be made of a material with a faster ingress rate than the remainder of the permeable layer 102 surrounding the absorbent layer 136. For example, the area of the permeable layer 102 around the absorbent dam 114 may have an ingress rate 2-4× faster than the ingress rate of the area of the permeable layer 102 not immediately above or surrounding the absorbent dam 114.

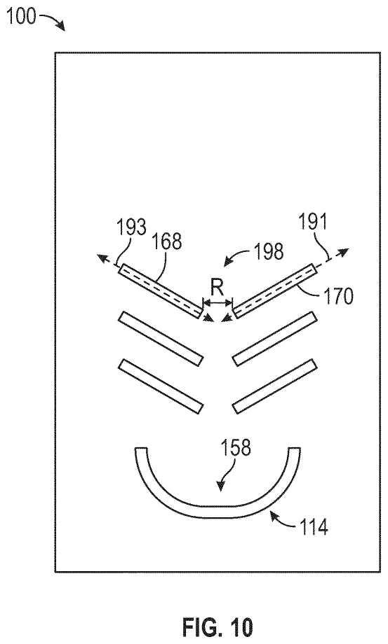

In some embodiments, the absorbent dam 114 includes one or more contiguous segments 190, 192, 194 (as shown in FIG. 2). The segments 190, 192, 194 may form a contiguous shape, such as a semi-circle or other rounded shape, as shown in FIG. 2. The first segment 194 may extend along a first axis 193. The first axis may extend substantially parallel to the impermeable layer fourth edge 132 (as shown in FIG. 2). However, it is understood that in some embodiments the first axis 193 does not extend substantially parallel to the impermeable layer fourth edge 132 (as shown in FIG. 10). The second segment 192 may extend along a second axis 191. The second axis 191 may be substantially parallel to the impermeable layer first edge 122 (e.g., extending from the first segment 194 at an angle 195 of 85-95°). However, it is understood that in some embodiments the second axis 191 does not extend substantially parallel to the impermeable layer first edge 122. The second axis 191 may be angularly separated from the first axis by an angle 195 between 45-135°. The third segment 190 may extend substantially parallel to the impermeable layer second edge 124 (e.g., extending from the first segment 194 at an angle of 85-95°). The third segment 190 may extend at an angle (e.g., 0-135°) from the first segment 194. The second segment 192 may extend from the first segment 194 at an angle (e.g., 0-135°). In some embodiments, the connection between the first segment 194 and the second segment 192 is angular. In some embodiments (as shown in FIG. 2), the connection between first segment 194 and the second segment 192 forms a curved connection 197. For example, the curved connection 197 may couple the first segment 194 and the second segment 192 such that the first segment 194 and the second segment 192 are contiguous in forming the absorbent dam 114. As shown in FIG. 2, the curved connection 197 may have a radius such that the first segment 194 and the second segment 192 extend tangentially from the curved connection 197. In some embodiments, the curved connection 197 has an arc angle of 45-135°. However, in other embodiments, the first segment 194 and the second segment 192 do not extend tangentially from the curved connection 197. While the curved connection 197 is shown in FIG. 2, it should be understood that the first segment 194 and the second segment 192 may be contiguously coupled with an angular connection (e.g., the second segment 192 extending from the first segment 194 at an angle of 0-135°).

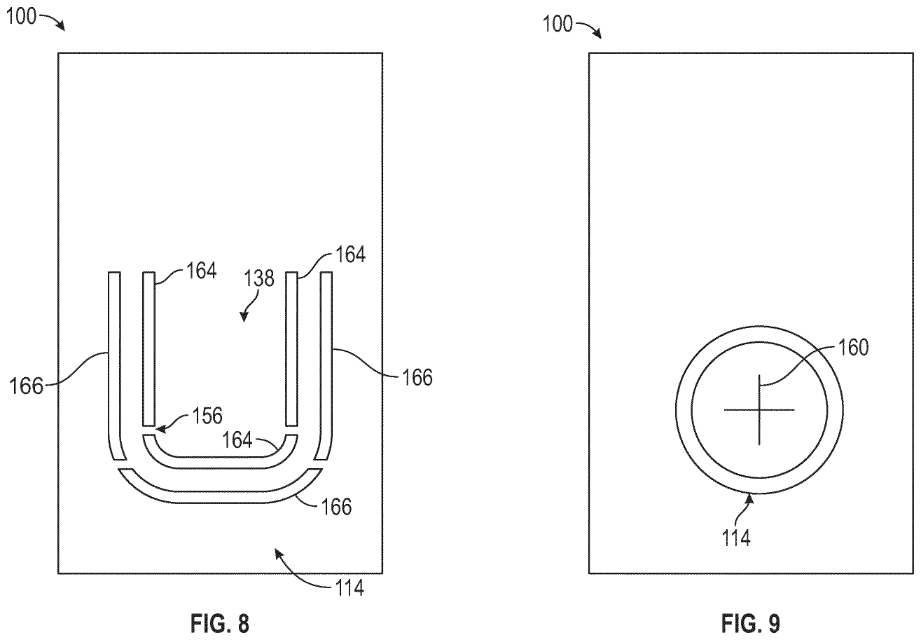

The absorbent dam 114 may be formed by a single contiguous dam of material, such as shown in FIGS. 1-7. However, alternative embodiments exist in which the absorbent medical sheet assembly 100 includes a plurality of noncontiguous absorbent dams 114 and/or alternative shapes to contain and/or control the flow of expelled biological waste. For example, as shown in FIGS. 8-10, alternative configurations of the absorbent dam 114 are illustrated. FIG. 8 illustrates a plurality of noncontiguous absorbent dams 114 with one or more breaches 156 between the noncontiguous absorbent dams 114 to control a flow of expelled biological waste away from the region 138. The one or more breaches 156 between the noncontiguous absorbent dams 114 may be used to control the flow of the expelled biological waste away from the region 138, rather than allowing the expelled biological waste to pool. In this way, the expelled biological waste may not overflow the absorbent dam 114. The one or more breaches 156 between the noncontiguous absorbent dams 114 may be used to discharge the expelled biological waste to a specific location, such as a collection device (e.g., a bag, bucket, bedpan).

The noncontiguous absorbent dams 114 shown in FIG. 8 include inner absorbent dams 164 and outer absorbent dams 166. In some embodiments, the permeable layer 102 around the inner absorbent dams 164 may have perforations for increasing the ingress rate of the expelled biological waste to the inner absorbent dams 164. The permeable layer 102 surrounding the outer absorbent dams 166 may have perforations as well. The perforations in the permeable layer 102 around the outer absorbent dams 166 may be smaller relative to the perforations in the permeable layer 102 around the inner absorbent dams 164, such that the permeable layer 102 surrounding the inner absorbent dams 164 has a faster ingress rate of the expelled biological waste than the permeable layer 102 surrounding the outer absorbent dams 166. In some embodiments, the permeable layer 102 around the outer absorbent dams 166 has no perforations. In such a manner, the outer absorbent dams 166 may act as a backup to absorb any excess biological waste that the inner absorbent dams 164 was unable to absorb. While the breach 156 is shown as a break between two noncontiguous inner absorbent dams 164, it is understood that the one or more breaches 156 may also include a section of the absorbent dam 114 with lesser height in the saturated state (e.g., by using less absorbent material at the one or more breaches 156).

FIG. 9 illustrates a fully enclosed absorbent dam 114. FIG. 9 illustrates the absorbent dam 114 as a circle, however, any closed shape may be used. For example, in some embodiments, the absorbent dam 114 is a square, a rectangle, a trapezoid, an oval, an irregular polygon, a rhombus, or a combination thereof. In some embodiments, the absorbent medical sheet assembly 100 includes an alignment marking 160 on the permeable layer 102 to aid in placement of the patient 150. The alignment marking 160 is shown as a crosshairs graphic, but it should be understood that the alignment marking 160 may include any graphical representation, of any color, in any location to aid in placement. For example, the alignment marking 160 may outline the location of the absorbent dam 114, include text with instructions for placement of the patient 150, have a shape of a patient in an ideal location for maximum absorption, and/or include color coding (e.g., green locations for placement, red for locations not to place the patient 150, such as on the edge of the absorbent medical sheet assembly 100). As shown in FIG. 10, angled absorbent dams 168 may be used to prevent the expelled biological waste or stool from traveling up a back of the patient 150. The additional angled absorbent dams 168 are angled so as to direct the expelled biological waste along a central axis of the absorbent medical sheet assembly 100 which leads to a central location 158. In some embodiments, the absorbent medical sheet assembly 100 includes one or more absorbent dams 114 along (or proximate to) the outer absorbent dam edges 170. As shown in FIG. 10, the noncontiguous absorbent dams are separated by a separation distance R 198 that is 0.1-0.4× the permeable layer width A 172.

In some embodiments, the absorbent dam 114 may include odor control to improve the comfort of the patient 150. For example, the absorbent dam 114 may be treated with a fluid solution with odor-neutralizing agents such as activated carbon, zeolites, baking soda, citric acid, and/or other derivatives therefrom. The treatments can include embedding the odor-neutralizing agents (e.g., activated carbon and/or zeolites) within the structure of the absorbent dam 114 and/or applying the odor-neutralizing to the absorbent dam 114 (e.g., baking soda and/or citric acid). The absorbent dam 114 may also be treated with fragrance or deodorants for reducing odor before, during, and/or after absorption. Some odor-neutralizing agents may be moisture-activated such that the odor-neutralizing properties are exhibited when the absorbent dam 114 is saturated.

Additionally or alternatively, the absorbent dam 114 may include antimicrobial properties to improve the hygiene of the patient 150. For example, the absorbent dam 114 may be treated with antimicrobial agents such as silver ions, zinc oxide, quaternary ammonium compounds, or a combination thereof. These compounds may inhibit bacterial or fungal growth. Other antimicrobial compounds that may be used to treat the absorbent dam 114 and reduce bacterial and/or fungal growth may include tea tree oil, aloe vera, chitosan, and/or a combination thereof.

As shown in FIGS. 4 and 5, the absorbent medical sheet assembly 100 has an unsaturated sheet height F 186 that is 0.5-2 inches. The absorbent dam 114 has an unsaturated dam height G 182 that is 0.1-0.8× the unsaturated sheet height F 186. The absorbent medical sheet assembly 100 has a saturated sheet height I 188 that is 1.5-5× larger than the unsaturated sheet height F 186. The absorbent dam 114 has a saturated dam height 184 that is 1.5-5× larger than the unsaturated dam height G 182.

The absorbent dam 114 may be positioned substantially in the middle of the permeable layer 102 relative to the permeable layer width A 172. The absorbent dam 114 may be located at a distance J 196 from the permeable layer first edge 104. The distance J 196 may be 0.1-0.5× the permeable layer width A 172. By positioning the absorbent dam 114 at the distance J 196 from the permeable layer first edge 104, the absorbent medical sheet assembly 100 is able to more easily roll widthwise for disposal of the absorbent medical sheet assembly 100 after use. The distance J 196 also allows for runoff from the absorbent dam 114 without flowing onto the support surface 128.

### III. Overview of Hand Covering

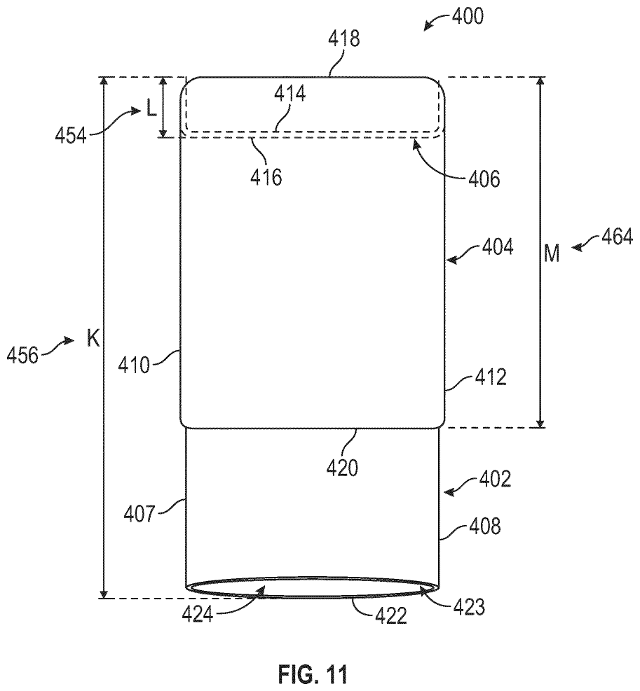

In a second implementation of the systems and methods described herein (as shown in FIGS. 11-20), a hand covering 400 (e.g., an absorbent mitten, sleeve, and/or glove) having an interior gusset (e.g., tab or handle) and external, rigid scraper coupled thereto may be used during the cleaning of biological waste (e.g., biological fluids, expelled waste, eliminated waste, expelled biological wastes, expelled biological waste) from a patient to prevent and/or contain the spread of biological waste during cleaning and disposal. In at least one implementation, the systems and methods described herein may be used in a medical/care facility by a user (e.g., a nurse, caretaker, assistant, or medical personnel) for cleaning a patient of the medical/care facility. As used herein, biological waste may include, but is not limited to, fecal matter (solid or liquid), urine, blood, sweat, exudate, and/or saliva.

The hand covering 400 may include an impermeable liner 402. The impermeable liner 402 acts as an impermeable barrier between a user (e.g., medical personnel) and biological waste. This impermeable barrier allows a user to use their hand for cleaning the biological waste off of a patient, particularly when the biological waste is within the patient's crevices and/or folds (e.g., in the patient's buttocks and/or groin area). The hand covering 400 allows the user to scoop and wipe (or otherwise remove) the biological waste with their hand without concern of contaminating themselves with the biological waste. In some embodiments, the impermeable liner 402 also provides a benefit of containing any cleaned biological waste during disposal of the hand covering 400. As will be described in detail below, the hand covering 400 may be turned inside out during disposal. By turning the hand covering 400 inside out during disposal, the cleaned biological waste is trapped within the impermeable liner 402, thus preventing spreading of the biological waste when disposing of the hand covering 400. As such, the impermeable liner 402 of the hand covering 400 may serve dual functionality: (1) protecting the user's hand during cleanup of the biological waste from ingress of biological waste into the hand covering 400 and (2) protecting the environment during disposal of the biological waste from egress of the biological waste out of the hand covering 400.

The impermeable liner 402 may be made of nitrile rubber, latex, polyvinyl chloride, chloroprene, polyethylene, polyurethane, butyl rubber, and/or ethylene vinyl acetate. Indeed, the impermeable liner 402 may be made of any impermeable, flexible material.

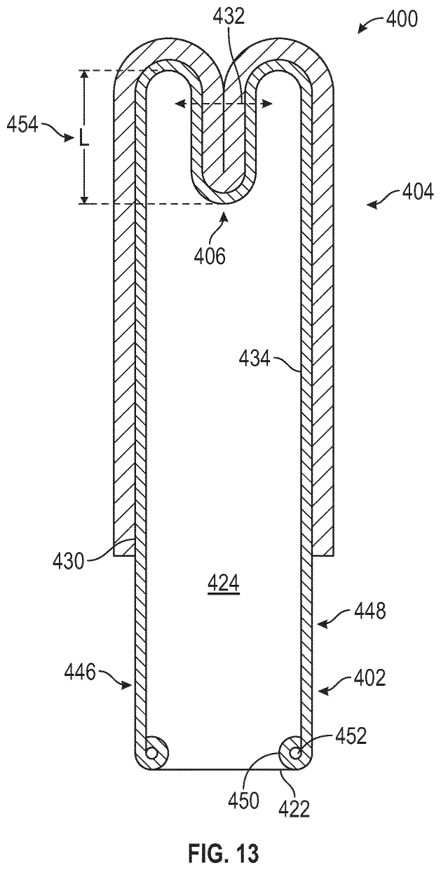

The impermeable liner 402 includes a liner interior surface 434 (as shown in FIG. 13). The liner interior surface 434 defines a liner cavity 424, into which the user's hand is placed during use of the hand covering 400. The impermeable liner 402 is configured to resist the transmission of fluid and/or solids between the liner interior surface 434 of the hand covering 400 and an exterior of the hand covering 400. In at least one embodiment, the liner cavity 424 may be a single cavity into which the user's hand is placed, without partitions (e.g., for fingers).

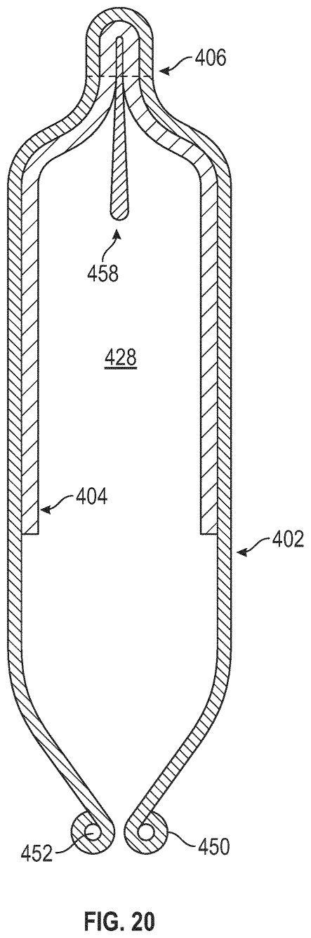

The impermeable liner 402 may form, in part, a gusset 406. The gusset 406 may be used by the user when removing the hand covering 400 such that hand covering 400 is turned inside out during removal. For example, the user may grasp the gusset 406 during removal of the hand covering 400. In this way, the biological waste cleaned with the hand covering 400 is contained within a hand covering cavity 428 of the inside-out hand covering 400 (as shown in FIG. 20). For example, when the hand covering 400 is in a first configuration (e.g., a cleaning configuration), as shown in FIGS. 11-20, the liner interior surface 434 defines the liner cavity 424 for the user's hand. In the first configuration, the cleaned biological waste is on the exterior of the hand covering 400. The hand covering 400 may also have a second configuration (e.g., a disposal configuration), as shown in FIG. 20, which is inside out relative to the first configuration. In the second configuration, the liner interior surface 434 forms an impermeable exterior surface with the biological waste in the hand covering cavity 428. The gusset 406 may be formed by joining a liner first face 446 (shown in FIG. 13) to the liner second face 448 (shown in FIG. 13) at a distance L 454 from a third liner edge 416.

In some embodiments, the hand covering 400 may include an elastic retention cord 452 (e.g., a bungee, an adjustable tension cord, drawstring, or elastic cuff string) for reducing the circumference of the fourth edge 422, which defines a first opening 423 of the hand covering 400 into the liner cavity 424. The elastic retention cord 452 may constrict the diameter of the first opening 423 defined by the fourth edge 422, thereby sealing the impermeable liner 402 against the user (e.g., at the wrist, forearm, upper arm) in the first configuration during cleaning of the biological waste. This prevents biological waste from entering the liner cavity 424 from the first opening 423 defined by the fourth edge 422. In a similar fashion, the elastic retention cord 452 may create a seal in the second configuration of the hand covering 400 during disposal of the hand covering 400 For example, the elastic retention cord 452 may be made of an elastic material with an extended length that is equal to or near a length of the fourth edge 422, thus allowing the fourth edge 422 to be opened to its largest diameter (e.g., the length of the fourth edge 422). The elastic retention cord 452 may have a non-extended length such that the non-extended length of the first opening 423 formed by the fourth edge 422 is less than the largest possible length of the fourth edge 422. In some embodiments, the non-extended length of the fourth edge 422 is nearly completely closed, causing the hand covering cavity 428 in the second configuration to be nearly closed, effectively sealing in any cleaned biological waste within the hand covering cavity 428.

The impermeable liner 402 may have a cuff 450 that defines a channel within which the elastic retention cord 452 is contained. The cuff 450 may be formed by folding the impermeable liner 402 over on itself and joining an edge of the cuff 450 to the impermeable liner 402. The elastic retention cord 452 may be placed within the cuff 450 during the folding or snaked into the channel defined by the cuff 450 after the cuff 450 is made.

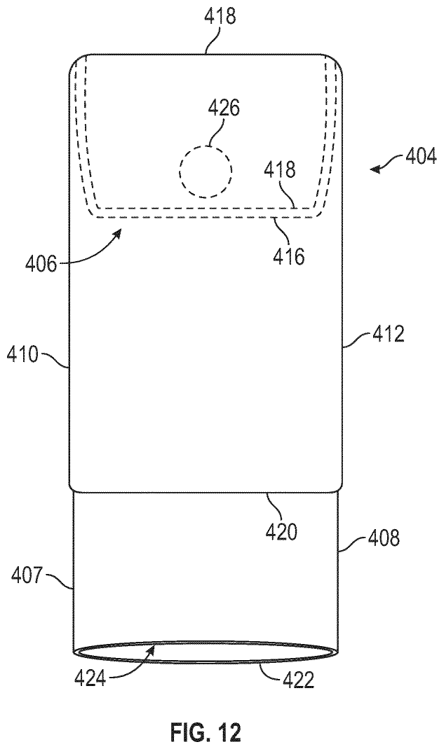

As shown in FIG. 12, the gusset 406 may include one or more features (e.g., finger hold 426) for facilitating removal of the hand covering 400. In FIG. 12, the gusset 406 is shown with the finger hold 426 (e.g., a hole through the layers of the hand covering 400) positioned thereon for the user to place a finger through when removing the hand covering 400. The finger hold 426 may be placed at any location on the gusset 406. While the finger hold 426 is shown as an aperture through which to place a finger, it should be understood that the finger hold 426 may alternatively be a handle, slit, tab, projection, or any other feature which facilitates gripping of the gusset 406. The finger hold 426 may be formed by the gusset 406 or may extend separately from the gusset 406.

As described above, the gusset 406 may be used during removal of the hand covering 400 to adjust the hand covering 400 from the first configuration to the second configuration.

Forming the impermeable liner 402 may include forming a sleeve of impermeable material, sealing the third liner edge 416, creating the gusset by sealing the liner first face 446 to the liner second face 448 at the distance L 454 from the third liner edge 416. This results in the impermeable liner 402 in the second configuration. The impermeable liner 402 may then be pulled over itself such that the gusset 406 extends within the liner cavity 424, thus resulting in the impermeable liner 402 in the first configuration. In an alternative embodiment, the gusset 406 is formed by a second material coupled to (e.g., by sewing, welding, adhering) the impermeable liner 402 (e.g., at the third liner edge 416) and extending into the liner cavity 424.

The impermeable liner 402 may have a liner length K 456. The liner length K 456 may be 6-30 inches. The distance L 454 is 0.1-0.25× the liner length K 456.

The impermeable liner 402 may form, at least in part, a body of the hand covering 400. The impermeable liner 402 may include a first liner edge 407, a second liner edge 408 opposite the first liner edge 407, a third liner edge 416, and/or a fourth edge 422 opposite the third liner edge 416. In an embodiment, the impermeable liner 402 may be a sleeve defined by the first liner edge 407, the second liner edge 408, the third liner edge 416, and/or the fourth edge 422. The impermeable liner 402 is closed on one end (e.g., at the third liner edge 416) and open at an opposing end (e.g., the fourth edge 422). The liner first face 446 extends from a first portion of the third liner edge 416 to the fourth edge 422. The liner second face 448 extends from a second portion of the third liner edge 416 to the fourth edge 422.

The hand covering 400 may include an absorbent sheet 404. The absorbent sheet 404 may be configured to absorb/clean biological waste on the patient, particularly within the crevices and/or folds of the user (e.g., the buttocks and/or groin). The absorbent sheet 404 may be configured to absorb fluids such as liquid stool and/or urine. For increased absorption, the absorbent sheet 404 may be made of a non-woven material such as polyester (PET), lyocell, polypropylene, cotton, cellulose pulp, bamboo fiber, flax fiber, superabsorbent polymers, polyamide, polylactic acid sheet, hemp fiber, or a combination thereof. In some embodiments, the absorbent sheet 404 may have a moisture-wicking layer and an absorption layer (e.g., a superabsorbent polymer). The absorbent sheet 404 is coupled to the impermeable liner 402.

The absorbent sheet 404 covers both the liner first face 446 and the liner second face 448 of the impermeable liner 402, thus providing a dual-sided cleaning hand cover. The user may use the dual-sided nature of the hand covering 400 to simultaneously clean both sides of a fold or crevice of the patient. The dual-sided nature of the hand covering 400 also allows the user to soil a first side of the hand covering 400 and then flip to the other unsoiled side of the hand covering 400 for additional cleaning.

The absorbent sheet 404 has a sheet length M 464 that is 0.25-0.75× the liner length K 456. By not extending the full length of the impermeable liner 402, the user is able to utilize all of the absorbent sheet 404 for cleaning, providing a benefit over traditional cleaning methods which require the user to hold a cleaning sheet (e.g., a towel), leading to unused portions of the cleaning sheet and risk of contact with the biological waste.

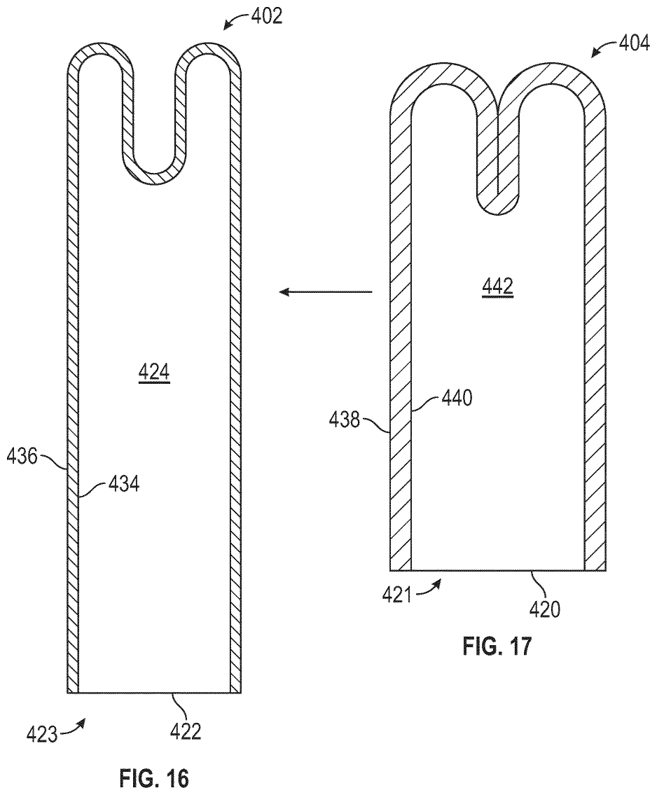

The absorbent sheet 404 may have a sheet interior surface 440 and a sheet exterior surface 438 when in the first configuration (as shown in FIG. 16). The sheet exterior surface 438 may be configured to aid in cleaning the biological waste and may have characteristics that facilitate cleaning (e.g., fluid absorbing, semi-abrasive for scrubbing dried biological waste, pre-moistened with a pH-neutralizing agent, softness for decreasing patient discomfort). By way of example, the absorbent sheet 404 may be impregnated with a pH-neutralizing agent. For example, in some embodiments the absorbent sheet 404 is pre-moistened with a chemical solution configured to neutralize acidic and/or basic biological waste during cleanup of the biological waste. The pH-neutralizing agent may be a chemical solution that includes one or more of sodium bicarbonate, citric acid, calcium carbonate, potassium bicarbonate, acetic acid, ammonium lactate, magnesium hydroxide, sodium citrate, trisodium phosphate, or a combination thereof. By pre-moistening the absorbent sheet 404 with the pH-neutralizing agent, the biological waste interacts with the pH-neutralizing agent during cleaning of the biological waste. The interaction of the pH-neutralizing agent and the acidic or basic biological waste results in the biological waste being pH-neutralized, leading to increased comfort of the patient.

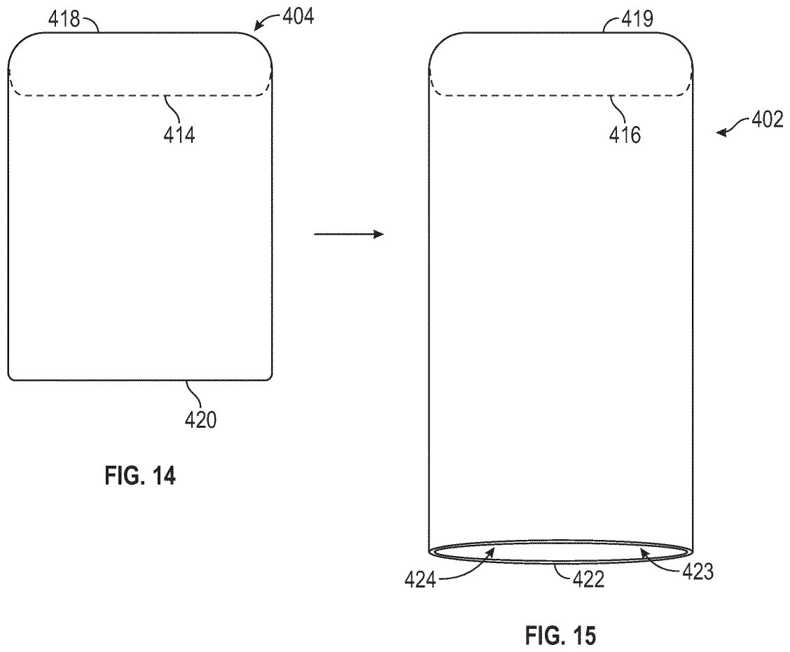

The sheet interior surface 440 may be coupled to the liner exterior surface 436 of the impermeable liner 402. The absorbent sheet 404 may be coupled to the impermeable liner 402 by one or more various methods, such as sewing, welding, and/or adhering. FIGS. 14 and 15 illustrate the coupling of the absorbent sheet 404 to the impermeable liner 402. FIG. 13 illustrates a side view of the hand covering 400 with the absorbent sheet 404 coupled to the impermeable liner 402.

In the first configuration, the sheet interior surface 440 defines a sheet cavity 442. The sheet cavity 442 may be configured to receive the impermeable liner 402. In coupling the impermeable liner 402 to the absorbent sheet 404 within the sheet cavity 442, the liner exterior surface 436 is coupled to the sheet interior surface 440 of the absorbent sheet 404. The liner exterior surface 436 may be coupled to the sheet interior surface 440 at one or more locations. For example, the liner exterior surface 436 may be coupled to the sheet interior surface 440 along an absorbent sheet edge 420 which defines a second opening 421 (as shown in FIG. 17). In some embodiments, the absorbent sheet 404 and the impermeable liner 402 are coupled at the line 432 through, for example, sewing, welding, or adhering.

The absorbent sheet 404 may have a third sheet edge 414. The third sheet edge 414 may be closed, similar to the third liner edge 416. In some embodiments, portions of the absorbent sheet 404 (e.g., the third sheet edge 414) extends into the gusset 406, such as shown in FIGS. 11-20. However, it should be understood that the absorbent sheet 404 need not extend into the gusset 406. In such embodiments, the absorbent sheet 404 may surround a portion of the impermeable liner 402 as an open sleeve.

In some embodiments, the impermeable liner 402 and/or the absorbent sheet 404 may include color indications (e.g., colored red) to communicate the contents of the hand covering 400. By way of a non-limiting example, the impermeable liner 402 may be red to communicate the presence of a biohazard (e.g., the biological waste). In at least one embodiment, the liner interior surface 434 is colored red such that in the second configuration (e.g., when the biological waste is contained within the hand covering 400) the hand covering 400 communicates to a user that there is biological waste in the hand covering cavity 428. Additionally or alternatively, the liner interior surface 434 (or any surface of the hand covering 400) may include text describing the use or contents of the hand covering 400.

The hand covering 400 may include a rigid scraper 458. The rigid scraper 458 may be configured to aid in removing biological waste (e.g., dried fecal matter) from the skin of the patient during cleaning of the patient. The rigid scraper 458 may extend from a third hand covering edge 418. However, it should be understood that the rigid scraper 458 may extend from any of a first hand covering edge 410, a second hand covering edge 412, and/or the third hand covering edge 418.

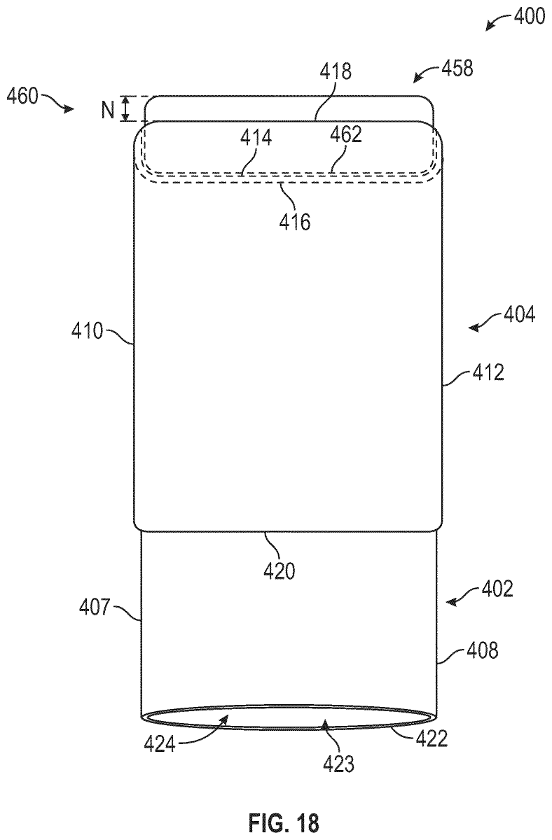

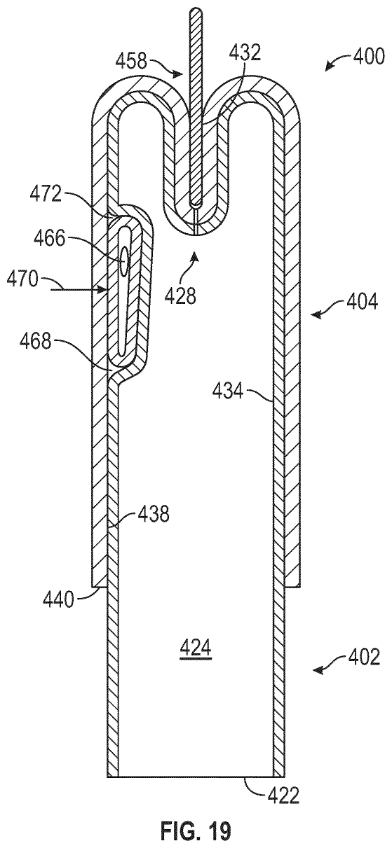

The rigid scraper 458 may be coupled to one or more of the impermeable liner 402 or the absorbent sheet 404. For example, as shown in FIGS. 18-20, the rigid scraper 458 may have a gusset edge 462 that extends into the gusset 406. The rigid scraper 458 is coupled to the absorbent sheet 404 within the gusset 406. The rigid scraper 458 may be coupled to the absorbent sheet 404 through sewing or adhesion. In some embodiments, in which the absorbent sheet 404 does not extend into the gusset 406, the rigid scraper 458 may be coupled to the impermeable liner 402 in the gusset 406. In a non-limiting embodiment, the rigid scraper 458 is coupled to the absorbent sheet 404 at the line 432 (as shown in FIG. 19) by sewing or adhesion. The rigid scraper 458 extends from an edge (e.g., the third hand covering edge 418) of the hand covering 400 by a distance N 460. The distance N 460 may be 0.01-0.1× the liner length K 456 of the hand covering 400.

The rigid scraper 458 may be a rigid or semi-rigid material with softened edges (e.g., non-sharp edges) that can follow the contours of the patient's skin without lacerating the patient. By way of non-limiting examples, the rigid scraper 458 may be made of silicone, rigid polypropylene, rubber-tipped material, ABS, polycarbonate, wood, bamboo, stainless steel, plastic, or a combination thereof.

The hand covering 400 may include a sealed, frangible enclosure 468 (e.g., a burst pouch) positioned between the impermeable liner 402 and the absorbent sheet 404. The sealed, frangible enclosure 468 may have a fluid solution 466 within that is configured to soften dried biological waste, provide a scent, and/or neutralize the pH of the biological waste. In some embodiments, the fluid solution 466 is a pH-neutralizing agent. The sealed, frangible enclosure 468 may be configured to break (e.g., burst, unseal, tear) in response to an applied force 470 to the sealed, frangible enclosure 468 satisfying (e.g., exceeding) a threshold force. By way of example, the sealed, frangible enclosure 468 may be configured to burst open and release the agent contained therein when the user squeezes the sealed, frangible enclosure 468 above a threshold force. In some embodiments, the threshold force is less than the average grip force of a human (e.g., 10-15 pounds). Upon breaking open, the fluid solution 466 may saturate the absorbent sheet 404 and facilitate cleaning (e.g., softening, pH neutralizing, providing odor) of the expelled biological matter. The frangible enclosure 468 may be positioned within a burst pouch cavity 472 that is defined by the sheet interior surface 440 and the sheet exterior surface 438.

In some embodiments, the hand covering 400 may be nested within a plurality of nesting hand coverings 400. By way of example, the user may place their hand within an innermost hand covering 400. Upon soiling the outermost hand covering 400, the user removes the outermost hand covering 400 and begins using the new outermost hand covering 400. This process may continue until the innermost hand covering 400 is the last hand covering 400.

### IV. Configuration of Example Embodiments

While this specification contains many specific implementation details, these should not be construed as limitations on the scope of what may be claimed but rather as descriptions of features specific to particular implementations. Certain features described in this specification in the context of separate implementations can also be implemented in combination in a single implementation. Conversely, various features described in the context of a single implementation can also be implemented in multiple implementations separately or in any suitable subcombination. Moreover, although features may be described as acting in certain combinations and even initially claimed as such, one or more features from a claimed combination can, in some cases, be excised from the combination, and the claimed combination may be directed to a subcombination or variation of a subcombination.

As utilized herein, the terms “substantially,” “generally,” “approximately,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the appended claims.

The term “coupled” and the like, as used herein, mean the joining of two components directly or indirectly to one another. Such joining may be stationary (e.g., permanent) or moveable (e.g., removable or releasable). Such joining may be achieved with the two components or the two components and any additional intermediate components being integrally formed as a single unitary body with one another, with the two components, or with the two components and any additional intermediate components being attached to one another.

It is important to note that the construction and arrangement of the various systems shown in the various example implementations is illustrative only and not restrictive in character. All changes and modifications that come within the spirit and/or scope of the described implementations are desired to be protected. It should be understood that some features may not be necessary, and implementations lacking the various features may be contemplated as within the scope of the disclosure, the scope being defined by the claims that follow. When the language “a portion” is used, the item can include a portion and/or the entire item unless specifically stated to the contrary.

Also, the term “or” is used, in the context of a list of elements, in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

Additionally, the use of ranges of values (e.g., W1 to W2) herein are inclusive of their maximum values and minimum values (e.g., W1 to W2 includes W1 and includes W2), unless otherwise indicated. Furthermore, a range of values (e.g., W1 to W2) does not necessarily require the inclusion of intermediate values within the range of values (e.g., W1 to W2 can include only W1 and W2), unless otherwise indicated.

What is claimed is:

1. An absorbent medical sheet assembly comprising:
an impermeable layer;
a permeable layer coupled to the impermeable layer, the permeable layer in confronting relation to the impermeable layer and cooperating with the impermeable layer to define a cavity therebetween; and
an absorbent dam positioned between the impermeable layer and the permeable layer such that the cavity extends around an entirety of a periphery of the absorbent dam.

2. The absorbent medical sheet assembly of claim 1, wherein the absorbent dam has a first volume in an unsaturated state and a second volume in a saturated state, the second volume being larger than the first volume.

3. The absorbent medical sheet assembly of claim 1, wherein:
the absorbent dam comprises:
a first segment extending along a first axis, and
a second segment extending at least in part along a second axis; and
the second axis is angularly separated from the first axis by an angle between 45-135°.

4. The absorbent medical sheet assembly of claim 3, wherein:
the permeable layer is defined by:
a permeable layer width extending from a permeable layer first edge to a permeable layer second edge, and
a permeable layer length extending from a permeable layer third edge to a permeable layer fourth edge; and
the first segment has a dam width that is 0.2-0.8× the permeable layer width.

5. The absorbent medical sheet assembly of claim 4, wherein the second segment has a dam length that is 0.2-0.8× the permeable layer length.

6. The absorbent medical sheet assembly of claim 4, wherein the second segment extends from the first segment at an angle between 85-95°.

7. The absorbent medical sheet assembly of claim 3, wherein the first segment is contiguous with the second segment to form a curved connection between the first segment and the second segment.

8. The absorbent medical sheet assembly of claim 4, wherein the first segment is noncontiguous with the second segment, the second segment separated from the first segment by 0.1-0.4× the permeable layer width.

9. A hand covering comprising:
an impermeable liner comprising:
a liner interior surface defining a liner cavity within the impermeable liner,
a liner exterior surface,
a first liner edge defining a first opening into the liner cavity, and
a second liner edge opposite the first liner edge, wherein the second liner edge is sealed;
an absorbent sheet comprising:
a sheet interior surface defining a sheet cavity within the absorbent sheet,
a sheet exterior surface, and
a sheet edge defining a second opening into the sheet cavity,
wherein the impermeable liner is positioned within the sheet cavity, the sheet interior surface being coupled to at least a portion of the liner exterior surface; and
a gusset extending into the liner cavity.

10. The hand covering of claim 9, further comprising a rigid scraper coupled to the sheet exterior surface, the rigid scraper extending into the gusset.

11. The hand covering of claim 9, further comprising a sealed, frangible enclosure positioned between the impermeable liner and the absorbent sheet, the sealed, frangible enclosure comprising a fluid solution therein and adapted to break in response to an applied force exceeding a threshold force of 10-15 pounds.

12. The hand covering of claim 9, wherein the absorbent sheet is impregnated with a pH-neutralizing agent.

13. The hand covering of claim 9, wherein the absorbent sheet has a sheet length that is 0.25-0.75× a liner length of the impermeable liner.

14. An absorbent medical sheet assembly comprising:
an impermeable layer;
an absorbent layer coupled to the impermeable layer; and
an absorbent dam positioned on the absorbent layer, the absorbent dam comprising:
a first segment extending along a first axis, and
a second segment extending at least in part along a second axis, the second axis angularly separated from the first axis, the first segment and the second segment defining a region therebetween configured to retain biological waste.

15. The absorbent medical sheet assembly of claim 14, wherein the first segment is noncontiguous with the second segment and defines a first breach therebetween.

16. The absorbent medical sheet assembly of claim 15, wherein the absorbent dam further comprises a third segment extending at least in part along a third axis, the third axis angularly separated from the first axis, the first segment being noncontiguous with the third segment and defining a breach therebetween.

17. The absorbent medical sheet assembly of claim 15, wherein the absorbent dam is a first absorbent dam, and the absorbent medical sheet assembly further comprises a second absorbent dam distal to the first absorbent dam and configured to receive biological waste through the first breach from the region.

18. The absorbent medical sheet assembly of claim 14, further comprising:
a first angled absorbent dam positioned on the absorbent layer; and
a second angled absorbent dam positioned on the absorbent layer a distance away from the first angled absorbent dam, the first angled absorbent dam and the second angled absorbent dam configured to direct the biological waste therebetween and into the region between the first segment and the second segment.

19. The absorbent medical sheet assembly of claim 18, wherein the second angled absorbent dam extends from the first angled absorbent dam at an angle between 45-135°.

20. The absorbent medical sheet assembly of claim 14, wherein the first segment is contiguous with the second segment to form a curved connection between the first segment and the second segment.
